(19) United States
(12) Patent Application Publication
(10) Pub. No.: US 2015/0151012 A1
(43) Pub. Date: Jun. 4, 2015

(54) DISINFECTION APPARATUS

(71) Applicant: KONINKLIJKE PHILIPS N.V., EINDHOVEN (NL)

(72) Inventor: Arnd Ritz, Heinsberg (DE)

(21) Appl. No.: 14/405,517

(22) PCT Filed: Jun. 18, 2013
(86) PCT No.: PCT/IB2013/054981

§ 371 Date: Dec. 4, 2014

Related U.S. Application Data
(60) Provisional application No. 61/664,833, filed on Jun. 27, 2012.

(51) Int. Cl. A61L 2/00; A61L 2/28; A61L 2/24

(52) U.S. Cl. CPC A61L 2/0058; A61L 2/24; A61L 2/28; A61L 2202/14

(57) ABSTRACT

The invention relates to an apparatus (100) and a method for the disinfection of skin areas, particularly for the disinfection of hands (H). Disinfection is achieved by application of a short IR light pulse emitted by at least one IR source(103) to the skin area. The parameters of the light pulse are chosen such that germs at the skin surface are killed while damaging of deeper skin layers is prevented.

### FIELD OF THE INVENTION

The invention relates to an apparatus and a method for the disinfection of skin areas, particularly for the disinfection of hands.

### BACKGROUND OF THE INVENTION

The WO 2006109897 A1 discloses a hand skin care apparatus with means for irradiating hands with IR rays, said means comprising for example a halogen lamp and/or a heated plate. Moreover, the apparatus comprises a low-level laser for projecting light beams onto a hand, in order to photoactivate beneficial reactions in the body.

### SUMMARY OF THE INVENTION

It was an object of the present invention to provide means that allow for a comfortable and efficient disinfection of skin areas, particularly of the hands of a user.

This object is achieved by an apparatus according to claim 1 and a method according to claim 2. Preferred embodiments are disclosed in the dependent claims.

According to its first aspect, the invention relates to an apparatus for the disinfection of skin areas, particularly for the disinfection of the skin of hands. The apparatus comprises the following components:
A chamber into which the skin area (or the part of the body with the skin area) can be placed during the treatment and which will in the following be called “disinfection chamber” for the purposes of reference.
An IR (infrared) source for exposing a skin area in the disinfection chamber to at least one pulse of IR light.

The disinfection chamber may in general be some volume for accommodating the skin to be disinfected. Preferably, the disinfection chamber is a cavity that it is open from (at least) one side for the introduction of the object to be disinfected.

Moreover, it should be noted that the term “disinfection” shall refer to any process that damages and/or kills microorganisms on the skin surface, particularly germs. Preferably, the “disinfection” shall lead to a measurable decrease of the number of germs on the respective skin area, for example induce more than a 1-log reduction (i.e. a reduction by a factor 0.1), preferably induce more than a 3-log reduction, most preferably induce more than a 4-log reduction.

The invention further relates to a method for the disinfection of skin areas, particularly of the hands, said method comprising the exposure of a skin area to at least one pulse of IR light. The method may particularly be carried out with an apparatus of the kind described above.

The apparatus and the method are based on the same inventive concept, i.e. the disinfection of skin areas by (short) pulses of IR light. Explanations and definitions provided for the apparatus are therefore also valid for the method and vice versa.

The apparatus and the method allow for an effective and comfortable disinfection of skin areas by exploiting short pulses of IR light, which have two main advantages: Firstly, a high radiation power and surface temperature (for killing germs) can be applied to the skin surface while the total radiation energy is kept sufficiently small to prevent damage of deeper skin layers. Secondly, application of just one or a few IR pulses makes the duration of the total disinfection procedure very short. This increases the comfort for the user and hence also overall safety because a proper disinfection will no longer be neglected due to its inconvenience or long duration.

In the following, various preferred embodiments of the invention will be described that relate to both the apparatus and the method described above.

The power delivered by the IR light pulse to the skin area to be disinfected should be high enough to enable rapid heating of the surface to damage or kill germs. Preferably, the energy density (here defined as: energy deposited per surface area) delivered to skin area by the pulses is, at any position within the disinfection chamber at which skin area to be disinfected can be disposed,
higher than about 0.1 J/cm² and/or lower than about 10 J/cm²,
higher than about 0.5 J/cm² and/or lower than about 5 J/cm², and/or
higher than about 1 J/cm² and/or lower than about 3 J/cm².

As an example, the energy density may range between about 0.5 J/cm² and about 3 J/cm².

By definition, a “pulse” of IR light shall have the duration of less than about five seconds. Preferably, the pulse duration is
longer than about 0.1 ms and/or shorter than about 1 s,
longer than about 0.5 ms and/or shorter than about 200 ms,
longer than about 1 ms and/or shorter than about 30 ms, and/or
longer than about 3 ms and/or shorter than about 10 ms.

These short pulse durations are particularly appropriate in combination with the aforementioned values for the total energy delivered per unit area to prevent sensation of pain and damage of deeper skin layers.

By definition, IR light has a wavelength between about 0.75 μm and about 1000 μm. In the context of the present invention, the wavelength of the applied IR light preferably is
longer than about 1.5 μm and/or shorter than about 30 μm,
longer than about 2 μm and/or shorter than about 12 μm, and/or
longer than about 2.5 μm and/or shorter than about 4 μm.

A single IR light pulse with appropriate parameters may already suffice to disinfect some skin area. It is however also possible to apply a series of several IR light pulses in order to achieve a better disinfection and/or to allow for a relative movement between the body part to be disinfected and the IR source such that a more uniform irradiation can be achieved. Moreover, distributing the delivered energy over several pulses may help to avoid heat damages of deeper skin layers.

In the aforementioned embodiment, the temporal distances (pauses) between subsequent IR light pulses preferably last
longer than about 1 ms and/or shorter than about 5 s,
longer than about 10 ms and/or shorter than about 1 s, and/or
longer than about 100 ms and/or shorter than about 500 ms.

The IR light pulse may have (approximately) homogeneous characteristics throughout the disinfection chamber, resulting in spatially uniform characteristics on the disinfected skin area. In a preferred embodiment, the IR light pulse may on the contrary be applied to the skin area with spatially inhomogeneous characteristics. Thus it is possible to treat different skin areas differently, allowing to adapt the IR treatment to the local requirements. For example, areas that are expected to carry higher concentrations of germs may be treated more intensely than other areas, or sensitive skin areas may be treated more cautiously. The characteristics which can vary spatially may for example comprise the intensity, the irradiance, the wavelength and/or the pulse duration and total pulse energy density of the applied IR light.

In general, the IR source may be any generator of infrared radiation with the required parameters. In particular, the IR source may comprise an LED (single LED or LED array), a flashlight, and/or an IR laser.

It is also within the scope of the present invention that the beam of the IR source is moved within the volume of the disinfection chamber, resulting in a movement of the irradiated zone across the skin area in the disinfection chamber. The pulses applied to an element of the surface may thus be created by scanning the beam of a continuously working radiation source over the surface under treatment. E. g. a laser spot may be scanned over the area of treatment in a 2-dimensional pattern or an oblong radiation pattern may be driven in a 1-dimensional movement over the surface. The total treatment area may be divided in segments which can be treated individually and repeatedly to generate a multiple pulse pattern for any point of the treatment area. The scanned radiation profile and the scanning action is provided by standard optical means known to those skilled in optical techniques.

In order to allow for a thorough disinfection of hands, the disinfection chamber may optionally comprise compartments for a separate accommodation of fingers. The disinfection chamber for hands may for example have the overall form of a glove with its fingers spread out. This ensures that also the areas where neighboring fingers contact each other are sufficiently disinfected.

In a further development of the invention, the disinfection may automatically be started if an object to be disinfected, for example a hand, enters a predetermined region of space. To this end, the apparatus may comprise at least one sensor for detecting the presence and/or the whereabouts of an object in the disinfection chamber. The sensor may for example comprise a light barrier, an ultrasound transducer, an IR detector or the like. With the help of such a sensor, the application of IR light in the detection chamber can favorably be controlled.

The aforementioned sensor in particular allows that the generation of an IR light pulse is initiated (only) when an object is detected in the disinfection chamber. This increases the comfort of the apparatus as a user does not need to press any button to start disinfection. Moreover, the generation of a series of light pulses can proceed as long as an object is detected in the disinfection chamber. A user can hence readily decide how long disinfection shall go on based on sensitive feedback; if for example the warming of the hand becomes uncomfortable, it simply suffices to withdraw the hand from the disinfection chamber.

### BRIEF DESCRIPTION OF THE DRAWINGS

These and other aspects of the invention will be apparent from and elucidated with reference to the embodiments described hereinafter.

In the drawings:

FIG. 1 schematically shows a top view onto an apparatus for hand disinfection according to the present invention;

FIG. 2 shows a section through said apparatus along line II-II of FIG. 1;

FIG. 3 schematically shows the time course of pulses that may be applied in the apparatus of FIG. 1;

FIG. 4 schematically shows a top view onto an alternative apparatus for hand disinfection in which a light bar is scanned over the skin surface.

Like reference numbers refer in the Figures to identical or similar components.

### DETAILED DESCRIPTION OF EMBODIMENTS

Good hand hygiene is an important factor in the prevention of diseases in general life and it is especially indispensable in the area of hospitals and doctor's offices. This importance is reflected by the strong recommendations given to health care workers to follow special procedures of hand disinfection at all indications necessary.

While the bare costs of the materials used for alcoholic hand sanitation are relatively low, hand disinfection is expensive because of the time needed for being carried out according to the recommendations. According to these recommendations every hygienic hand antisepsis takes at least 30 seconds of hand treatment with antiseptic agents.

It has been counted that depending on the clinical department there are between 100 and 200 instances demanding hand disinfection from every health care worker a day. This means more than 100 times about 30 sec, in total about 1 to 2 hours of hand cleaning every day.

The consequence is that the compliance of hand hygiene is only about 20% of the necessary instances in hospitals as seen in tests.

This increases the rate of nosocomial infections (infections related to clinical treatment), such as pneumonia, sepsis or wound infection. For Germany it is estimated that there are about 500000 cases a year and that of these 10000 to 15000 people die of nosocomial infections a year.

So here is an urgent need for clean hands that is poorly met in the medical practice.

It is a purpose of the present invention to release the main obstacle for a better hand hygiene in hospitals, which is the high time consumption of today's hand disinfection methods.

In order to achieve this object, it is proposed to use short pulses of IR radiation brought to the skin, so that the thin surface layer of the skin, which is decisive for hand hygiene, acquires temperatures high enough to reduce the disease evoking germs on the hands of health care workers.

Heat conduction then spreads out the heat pulse into deeper layers of the skin, thereby flattening the temperature profile. So the deep laying sensors for temperature and pain only encounter a mild rise in temperature of a few degrees.

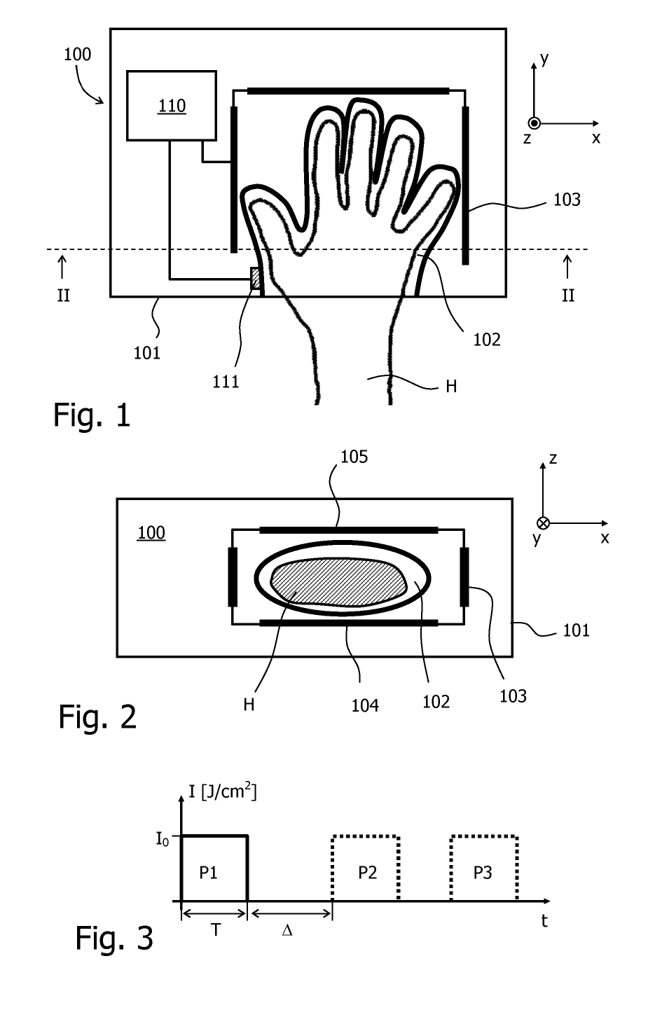

FIGS. 1 and 2 show schematically an apparatus 100 that is designed according to the above general principles. The apparatus 100 comprises a casing 101 with a recess or cavity 102, called “disinfection chamber” in the following, into which the hand(s) H of a user can be put. In the shown embodiment, the disinfection chamber 102 has roughly the form of a glove with its fingers spread out.

The apparatus 100 comprises at least one IR source 103, 104, 105 that directs its radiation onto the surface of the hand-like lumen of the disinfection chamber 102. The light source(s) 103, 104, 105 can be an array of LEDs or flash lamps or the beam of a laser. In either case the arrangement is made in such a way that the light energy is distributed evenly or with a predefined pattern over the inner surface of the disinfection chamber 102, either by the positioning of the individual light sources or by reflection and scattering means (not shown).

The IR sources 103, 104, 105 are coupled to a control unit 110 by which they are supplied with electrical power in order to initiate the emission of one or more IR light pulses.

FIG. 3 shows in this respect schematically the irradiance I caused by IR light pulses P1, P2, P3, . . . on the surface of an object in the disinfection chamber 102. The IR light is applied during at least one pulse P1 having a duration T of for example T=10 ms. The total energy density I₀ of the pulse has a mean value of about 1 J/cm². Optionally, a series of several pulses P1, P2, P3, . . . can be applied with intermediate pauses A (of the same or of varying lengths) of for example Δ=5 ms.

During application, a user puts his hands into the apparatus 100 with his fingers splayed out. A sensor 111 inside the apparatus may be used to detect the complete introduction of the hand(s) H, wherein this detection makes the control unit 110 release one or several pulses of IR radiation. As a result, the user feels a mild warming of his hands and recognizes that the disinfection has been done.

In a further embodiment, several sensors (or one more elaborate sensor) may be present to selectively control different parts of the disinfection chamber 102 separately.

The distribution of the IR pulse energy can also be adapted according to the skin thickness and germ distribution over the surface of the hands. It is also within the scope of the invention to apply several pulses or to apply individual pulses in different areas of the skin of the hands.

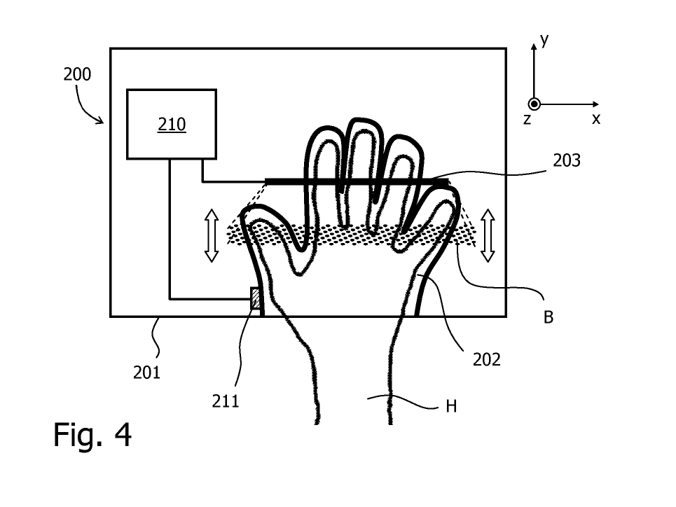

FIG. 4 shows an alternative apparatus 200 for hand disinfection with which the aforementioned approaches can be realized. The apparatus 200 is basically similar to that of FIGS. 1 and 2, so that it will not be described in detail again.

The difference with respect to the previous apparatus 100 is that there is a light source 203 which emits a movable beam into the disinfection chamber 202, resulting in a moving bar-shaped irradiated zone B on a hand H in the disinfection chamber 202 (a further, not visible light source may be located below the hand H). During a disinfection procedure, this irradiated zone B is scanned (in y-direction) over at least a part of the volume of the disinfection chamber 202 by appropriate means (e.g. mirrors) to apply a pulse of IR light to every desired position on the surface of the hand H. By repetitive scans, several pulses can be applied, too. Moreover, parameters like the energy density of the pulses can locally be varied across the skin surface by synchronizing the emission of the IR source 203 appropriately with the bar position.

In a modification of the above approach, the irradiated region may be a spot (instead of a bar) which is scanned in a two-dimensional pattern across the skin surface. This allows for an individual irradiation (pulse duration, pulse number, energy density etc.) of each position on the skin area to be disinfected.

Moreover, a variation of the intensity profile across the IR beam may be used to apply different irradiation parameters to different zones on a skin area.

The invention is not limited in its use to hospitals, but can in likewise be applied in e.g. the offices of doctors and dentists, in restaurants and canteens, in shops with unwrapped foodstuff, in schools and public buildings, in rest rooms or the like.

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

1. An apparatus for the disinfection of skin areas, particularly of the hands, comprising:
a disinfection chamber;
an IR source for exposing a skin area in the disinfection chamber to at least one pulse of IR light, said pulse having a duration of less than 5 seconds,
wherein the disinfection chamber further comprises a sensor for detecting the presence and/or the whereabouts of an object.

2. (canceled)

3. The apparatus according to claim 1,
characterized in that the energy density of the pulse at the skin area ranges is higher than about 0.1 J/cm² and/or lower than about 10 J/cm².

4. The apparatus according to claim 1,
characterized that the duration of the pulse is less than about 1 s and/or longer than about 0.1 ms.

5. The apparatus according to claim 1,
characterized in that the wavelength of the IR light ranges is longer than about 1.5 μm and/or shorter than about 30 μm.

6. (canceled)

7. The apparatus or the method according to claim 6,
characterized in that a pause between subsequent IR light pulses lasts longer than about 1 ms and/or shorter than about 5 s.

8. The apparatus according to claim 1,
characterized in that the IR light pulse is applied to the skin area with spatially inhomogeneous characteristics.

9. The apparatus according to claim 1,
characterized in that the IR source comprises at least one LED, a flashlight, and/or an IR laser.

10. The apparatus according to claim 1,
characterized in that the irradiation zone of the IR source is moved across the skin area in the disinfection chamber.

11. The apparatus according to claim 1,
characterized in the disinfection chamber comprises compartments for the separate accommodation of fingers to be disinfected.

12. (canceled)

13. The apparatus according to claim 1,
characterized in that the generation of an IR light pulse is initiated when an object is detected in the disinfection chamber.

14. A method of controlling an apparatus for the disinfection of skin areas, particularly of the hands, comprising:
a disinfection chamber;
an IR source for exposing a skin area in the disinfection chamber to at least one pulse of IR light, said pulse having a duration of less than 5 seconds,
wherein the disinfection chamber further comprises a sensor for detecting the presence and/or the whereabouts of an object,
wherein the method comprises:
detecting the presence and/or the whereabouts of an object in the disinfection chamber,
supplying the IR source with electrical power in order to initiate the emission of one or more IR light pulses.

15. A method according to claim 11, further comprising the step of controlling the IR source such that an irradiation zone of the IR source is moved across the object in the disinfection chamber.
